(19) United States
(12) Patent Application Publication
(10) Pub. No.: US 2023/0301773 A1
(43) Pub. Date: Sep. 28, 2023

(54) INTRAOCULAR LENSES WITH NANOSTRUCTURES AND METHODS OF FABRICATING THE SAME

(71) Applicant: Alcon Inc., Fribourg (CH)

(72) Inventors: Hao Zhang, Keller, Tx (US); Sumit Paliwal, Grapevine, Tx (US); John Janders, Fort Worth, Tx (US); Shridhar Natarajan, Arlington, Tx (US); Xin Hong, Fort Worth, Tx (US)

(21) Appl. No.: 18/187,283

(22) Filed: Mar. 21, 2023

Related U.S. Application Data
(60) Provisional application No. 63/363,828, filed on Apr. 29, 2022.
(60) Provisional application No. 63/269,719, filed on Mar. 22, 2022.
(60) Provisional application No. 63/364,813, filed on May 17, 2022.

(51) Int. Cl. A61F 2/16

(52) U.S. Cl. CPC A61F 2/1613; A61F 2240/004; A61F 2002/1681

(57) ABSTRACT

Certain aspects provide an intraocular lens (IOL) including a monolithic lens body having an anterior nanostructure assembly on an anterior outer surface of the monolithic lens body and a posterior nanostructure assembly on a posterior outer surface of the monolithic lens body, and one or more haptics coupled to the monolithic lens body.

### BACKGROUND

The human eye in its simplest terms functions to provide vision by transmitting light through a clear outer portion called the cornea, and focusing the image by way of a lens onto a retina. The quality of the focused image depends on many factors including the size and shape of the eye, and the transparency of the cornea and lens. When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an intraocular lenses (IOLs).

Although existing IOLs as well as methods and systems for manufacturing thereof may be acceptable, they also have certain shortcomings. Accordingly, there is a need for improvements to IOL designs and associated manufacturing techniques for complex optical designs.

### SUMMARY

Aspects of the present disclosure provide an intraocular lens (IOL), including a monolithic lens body having an anterior nanostructure assembly on an anterior outer surface of the monolithic lens body and a posterior nanostructure assembly on a posterior outer surface of the monolithic lens body, and one or more haptics coupled to the monolithic lens body.

Aspects of the present disclosure also provide a lens mold for forming an intraocular lens (IOL) by injection molding. The lens mold includes a first mold half having a first inner surface, the first inner surface corresponding to an external geometry of an anterior outer surface of an IOL to be formed, and a second mold half having a second inner surface, the second inner surface corresponding to an external geometry of a posterior outer surface of the IOL to be formed. The first inner surface has a first nanostructured pattern formed thereon, and the second inner surface has a second nanostructured pattern formed thereon.

Aspects of the present disclosure further provide a method for fabricating an intraocular lens (IOL). The method incudes fabricating a lens mold comprising a first mold half and a second mold half, and fabricating a single monolithic lens body using the lens mold by injection molding and thermal curing. The first mold half has a first nanostructured pattern formed on an inner surface of the first mold half, and the second mold half has a second nanostructured pattern formed on an inner surface of the second mold half.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is noted, however, that the appended drawings illustrate only some aspects of this disclosure and the disclosure may admit to other equally effective embodiments.

FIG. 1A depicts a top view of an intraocular lens (IOL), according to certain embodiments.

FIG. 1B depicts a side view of a portion of the IOL of FIG. 1A, according to certain embodiments.

FIG. 1C depicts an enlarged view of a portion of the IOL of FIG. 1A, according to certain embodiments.

FIG. 2 depicts example operations for fabricating an IOL with nanostructures, according to certain embodiments.

FIGS. 3A, 3B, 3C, and 3D illustrate various aspects of a lens mold and a substrate corresponding to the different stages of the operations of FIG. 2, according to certain embodiments.

FIG. 4 depicts example operations for forming a mold half, according to certain embodiments.

FIGS. 5A, 5B, and 5C illustrate various aspects of a lens mold and a substrate corresponding to the different stages of the operations of FIG. 4, according to certain embodiments.

FIG. 6 depicts example operations for forming a mold half, according to certain embodiments.

FIGS. 7A, 7B, 7C, 7E, and 7F illustrate various aspects of a lens mold and a substrate corresponding to the different stages of the operations of FIG. 6, according to certain embodiments.

FIG. 8 depicts an example system for designing, configuring, and/or forming an IOL, according to certain embodiments.

FIG. 9 depicts example operations for forming an IOL, according to certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

The embodiments described herein provide methods and systems for fabricating a single monolithic intraocular lens (IOL) having nanostructured patterns embossed onto on external surfaces of the IOL, by injection molding (e.g., conventional injection molding). In certain embodiments, the methods and the system include producing a plastic mold having a cavity therein, in which surfaces of the cavity have nanostructured patterns, and thus fabricating an IOL having nanostructured patterns on an external surface of the IOL when fabricated by injection molding using the plastic mold.

State-of-the art IOLs that include nanostructures on the external surface of the lens have alleviated visual difficulties and discomfort, such as glare, scary eye, and halo, as the refractive index and reflectivity of the lens can be modified by the nanostructures. However, nanostructures are currently fabricated separately, for example, by lithography processes, to be subsequently attached to an IOL that is fabricated using conventional molding. The embodiments described herein provide fabrication processes for fabricating a plastic mold that allows for fabrication of a single piece monolithic IOL having nanostructured patterns embossed onto the external surfaces of the IOL, using injection molding. Fabrication of a single piece monolithic IOL may decrease processing time and cost. In addition, the embodiments described herein do not require attaching nanostructures to an IOL, thereby removing any concerns in relation to nanostructures detaching from an IOL in the human eye.

An IOL with Nanostructures

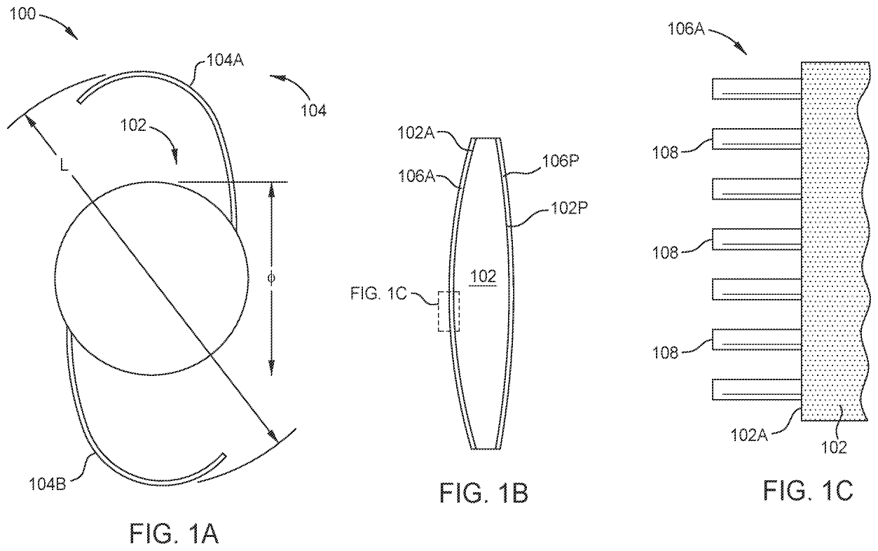

FIG. 1A illustrates a top view of an intraocular lens (IOL) 100, according to certain embodiments. FIG. 1B illustrates a side view of a portion of the IOL 100. FIG. 1C illustrates an enlarged side view of a portion of the IOL 100. The IOL 100 includes a lens body 102, having an anterior outer surface 102A and a posterior outer surface 102P, and a haptic portion 104 that is coupled to a peripheral, non-optic portion of the lens body 102. It is noted that the shape and curvatures of the lens body 102 are shown for illustrative purposes only and that other shapes and curvatures are also within the scope of this disclosure. For example, the lens body 102 shown in FIG. 1B has a bi-convex shape. In other examples, the lens body 102 may have a plano-convex shape, a convexo-concave shape, or a plano-concave shape.

The lens body 102 has a diameter ϕ of between about 4.5 mm and about 7.5 mm, for example, about 6.0 mm. The lens body 102 includes an anterior nanostructure assembly 106A on the anterior outer surface 102A, and a posterior nanostructure assembly 106P on the posterior outer surface 102P. In certain embodiments, the lens body 102 includes only one of the anterior nanostructure assembly 106A or the posterior nanostructure assembly 106 and the outer surface 102A, or 102P that does not have a nanostructure assembly formed thereon includes a diffractive structure to adjust a refractive index and/or reflectivity of the lens. In certain embodiments, as shown in FIG. 1B, the IOL 100 is a mono-focal IOL (with a single focal point) having no annular echelettes formed on the anterior outer surface 102A or the posterior outer surface 102P. In some other embodiments, the IOL 100 is a multi-focal IOL (with multiple focal points, e.g., bi-focal and tri-focal) having annular echelettes on the anterior outer surface 102A and/or the posterior outer surface 102P (not shown). In some other embodiments, the IOL 100 is an extended depth of focus (EDOF) IOL (with elongated focus) having annular echelettes on the posterior outer surface 102P (not shown).

As shown in FIG. 1C, the anterior nanostructure assembly 106A includes protrusions 108. The posterior nanostructure assembly 106P includes similar protrusions 108. Shapes, sizes, and density (e.g., spacing between adjacent protrusions 108) of the protrusions 108 are designed for the lens body 102 to provide a desired refractive index. For example, the protrusions 108 may have heights H of between about 30 nm and about 200 nm, widths of between about 30 nm and about 300 nm, and spacings between adjacent protrusions 108 of between about 30 nm and about 300 nm. The nanostructure assemblies 106A, 106P may further reduce reflectivity by between about 10% and about 90%, as compared to a lens body having no nanostructure assemblies on its anterior outer surface or posterior outer surface. In certain embodiments, as shown in FIG. 1B, the nanostructure assemblies 106A, 106P entirely cover the anterior outer surface 102A and the posterior outer surface 102P, respectively. However, in some other embodiments, the nanostructure assemblies 106A, 106P only partially cover the anterior outer surface 102A and the posterior outer surface 102P, respectively.

The lens body 102 and the nanostructure assemblies 106A, 106P may be fabricated as a single monolithic piece using a transparent, flexible, biocompatible lens forming material, such as modified poly (methyl methacrylate) (PMMA), modified PMMA hydrogels, hydroxy-ethyl methacrylate (HEMA), PVA hydrogels, other silicone polymeric materials, hydrophobic acrylic polymeric materials, for example, AcrySof® and Clareon®, available from Alcon, Inc., Fort Worth, Texas. The lens body 102 may have a refractive index n of between about 1.49 and about 1.56.

In certain embodiments, the anterior outer surface 102A and/or the posterior outer surface 102P of the lens body 102 may be fabricated of a biocompatible material (e.g., polymethyl methacrylate (PMMA), stiffer than the material of the remaining portions of the lens body 102.

The haptic portion 104 includes radially-extending struts (also referred to as “haptics”) 104A and 104B. The haptics 104A and 104B may be fabricated of biocompatible material, such as PMMA. The haptics 104A and 104B are coupled (e.g., glued or welded) to the peripheral portion of the lens body 102 or molded along with a portion of the lens body 102, and thus extend outwardly from the lens body 102 to engage the perimeter wall of the capsular sac of the eye to maintain the lens body 102 in a desired position in the eye. The haptics 104A and 104B typically have radial-outward ends that define arcuate terminal portions. The terminal portions of the haptics 104A and 104B may be separated by a length L of between about 6 mm and about 22 mm, for example, about 13 mm. The haptics 104A and 104B have a particular length so that the terminal portions create a slight engagement pressure when in contact with the equatorial region of the capsular sac after being implanted. While FIG. 1A illustrates one example configuration of the haptics 104A and 104B, any plate haptics or other types of haptics can be used.

Fabrication of an IOL with Nanostructures

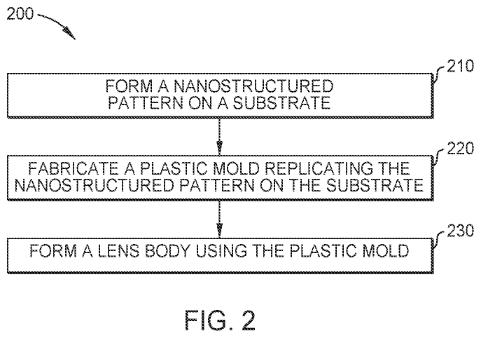

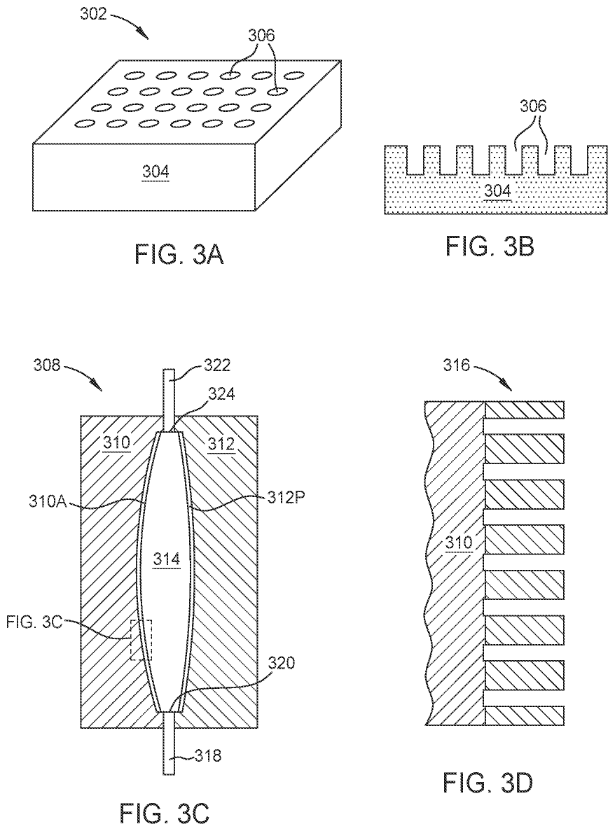

FIG. 2 depicts example operations 200 for fabricating an IOL with nanostructures. FIGS. 3A, 3B, 3C, and 3D illustrate various aspects of a lens mold and a substrate corresponding to the different stages of the operations 200. As such, FIGS. 2, 3A, 3B, 3C, and 3D are described together.

At step 210, a nanostructured pattern 302 is formed on a substrate 304 by standard micro/nano fabrication methods, as shown in an isometric view in FIG. 3A and a cross-sectional view in FIG. 3B. For example, first, a photolithography process is performed to spin on a photoresist on a substrate 304, and selected areas of the photoresist are exposed to light and developed. Second, the pattern of the photoresist is transferred to the substrate 304 by reactive ion etching. Then, the remaining photoresist is removed by plasma over-etching. The nanostructured pattern 302 may extend over an area of about 7 mm and about 7 mm of the substrate 304, and include an array of trenches 306 carved in the substrate 304 with heights of between about 30 nm and about 200 nm, widths of between about 30 nm and about 300 nm, and spacings between adjacent trenches of between about 30 nm and about 300 nm.

The term “substrate” as used herein refers to a layer of material that serves as a basis for subsequent processing operations and includes a surface to be cleaned. For example, the substrate may include glass, or one or more conductive metals, such as nickel, titanium, platinum, molybdenum, rhenium, osmium, chromium, iron, aluminum, copper, tungsten, or combinations thereof. The substrate can also include one or more materials comprising silicon, including materials associated with group IV or group III-V including compounds, such as Si, polysilicon, amorphous silicon, silicon nitride, silicon oxynitride, silicon oxide, Ge, SiGe, GaAs, InP, InAs, GaAs, GaP, InGaAs, InGaAsP, GaSb, InSb and the like, or combinations thereof. Furthermore, the substrate can also include dielectric materials such as silicon dioxide, organosilicates, and carbon doped silicon oxides. Further, the substrate can include any other materials such as metal nitrides, metal oxides and metal alloys, depending on the application.

Moreover, the substrate is not limited to any particular size or shape. The substrate can be a round wafer having a 200 mm diameter, a 300 mm diameter, a 450 mm diameter or other diameters. The substrate can also be any polygonal, square, rectangular, curved or otherwise non-circular workpiece, such as a polygonal glass, plastic substrate.

At step 220, a lens mold 308 replicating the nanostructured pattern 302 on the substrate 304 is fabricated, as shown in a cross-sectional view in FIG. 3C. The lens mold 308 may be formed of plastic material, such as polypropylene (PP) or polycarbonate (PC). As shown in FIG. 3C, the lens mold 308 includes a first mold half 310 and a second mold half 312. The first and second mold haves 310, 312 are coupled together (e.g., by being held together using pressure fitting, by one or more snaps or other retainers (not shown) attached to or integrally formed with the mold half 310, 312) to form a cavity 314 between the first and second mold halves 310, 312. Each mold half 310, 312 defines an inner surface 310A, 312P corresponding to the desired external geometry of the anterior outer surface 102A and the posterior outer surface 102P of the lens body 102, respectively. In the example shown in FIG. 3C, each mold half 310, 312 defines a concave inner surface, forming a bi-convex lens body 102.

On the inner surfaces 310A, 312P of each mold half 310, 312, a nanostructured pattern 316 replicating the nanostructured pattern 302 on the substrate 304 is formed, for forming the nanostructure assemblies 106A, 106P on the anterior outer surface 102A and the posterior outer surface 102P, respectively, of the lens body 102. For example, the nanostructured pattern 316 may include protrusions having heights of between about 30 nm and about 200 nm, widths of between about 30 nm and about 300 nm, and spacings between adjacent protrusions of between about 30 nm and about 300 nm.

In some embodiments, each mold half 310, 312 having the nanostructured pattern 316 is fabricated by directly depositing mold forming material onto the substrate using a focused ion beam, as described below in relation to operations 400. In some other embodiments, each mold half 310, 312 having the nanostructured pattern 316 is fabricated by soft lithography processes, as described below in relation to operations 600.

At step 230, a lens body 102 having nanostructure assemblies 106A, 106P is formed as a single monolithic piece using the lens mold 308 by injection molding and thermal curing. First, a lens forming material in liquid form, such as pre-polymer, is injected into the cavity 314 of the lens mold 308 (e.g., through a fill needle 318 from an opening 320), to a level sufficient to produce a lens body 102 of a desired geometry. In certain embodiments, a pre-polymer is a monomer, a polymer, or a polyvinyl alcohol (PVA) hydrogel.

The cavity 314 can be vented through a vent needle 322 disposed in an opening 324, which is open to the atmosphere or connected to a vacuum source such as a low pressure plenum (not shown). Second, the lens forming material within the lens mold 308 is cured by applying light (e.g., UV light) or heat to polymerize or cross-link the pre-polymer (e.g., thermal radiation) to form the solid lens body 102. Then, the solid lens body 102 is extracted from the lens mold 308.

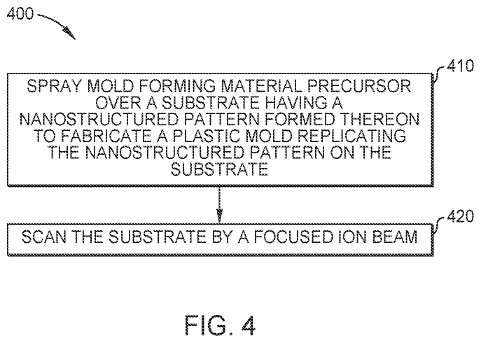

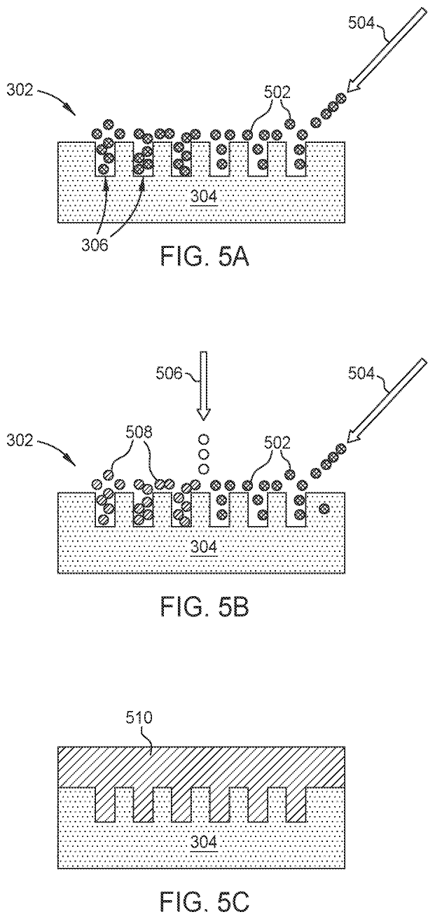

FIG. 4 depicts example operations 400 for forming each mold half 310, 312 (i.e., step 220) by a focused ion beam (FIB)-assisted chemical vapor deposition (CVD) process. FIGS. 5A, 5B, and 5C illustrate aspects of a lens mold and a substrate corresponding to the different various stages of the operations 400. As such, FIGS. 4, 5A, 5B, and 5C are described together.

At step 410, a mold forming material precursor 502 is sprayed from a precursor gas source 504 over the substrate 304 having the nanostructured pattern 302 formed thereon, as shown in FIG. 5A. The mold forming material precursor may be hydroxyl-groups-containing polypropylene (PP) precursor, such as Isotactic polypropylene graft copolymers, isotactic [polypropylene-graft-poly(methyl methacrylate)] (i-PP-g-PMMA) and isotactic [polypropylene-graft-polystyrene] (i-PP-g-PS) to fabricate a lens mold 308 of polypropylene (PP). The mold forming material precursor may be monomer bisphenol A (BPA) containing polycarbonate (PC) precursor. The mold forming material precursor 502 may adhere to a surface of the substrate 304 and inner walls of the trenches 306.

At step 420, the substrate 304 is scanned by a focused ion beam 506. In a typical FIB system, gallium ions are used to provide ion beams, although other ions and ion sources, such as multi-cusp or other plasma ion source, can be used. The ion beam 506 may decompose the mold forming material precursor 502 into different smaller components, including volatile components (not shown) and non-volatile components 508. The non-volatile components remain on the substrate 304 while the volatile components are pumped out by a pumping system (not shown).

With a continuous supply of the mold forming material precursor 502 from the precursor gas source 504 (step 410) and ion beam irradiation (step 420), the mold forming material, e.g., polypropylene (PP) or polycarbonate (PC) precursor, can be deposited on a desired area of the substrate 304 including the nanostructured pattern 302, forming a mold 510 replicating the nanostructured pattern 302 on the substrate 304. This mold 510 can be used as a mold half 310, 312 having a nanostructured pattern 316 (shown in FIGS. 3C and 3D).

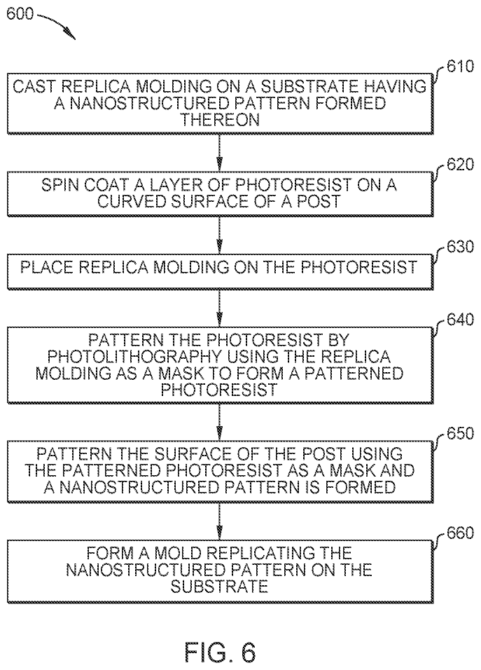

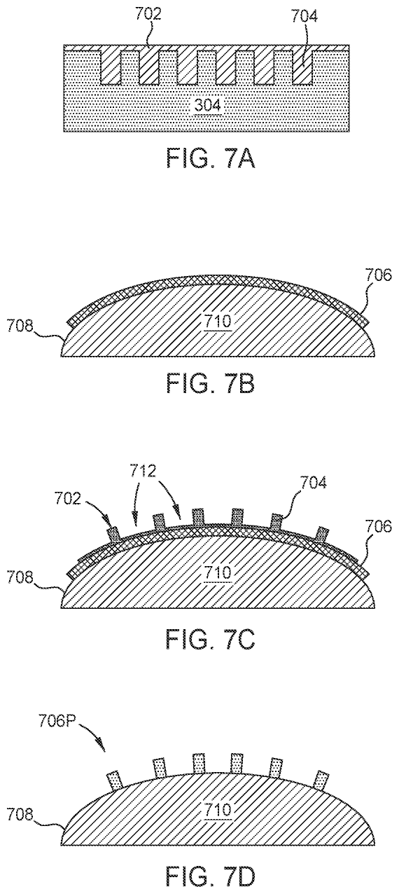

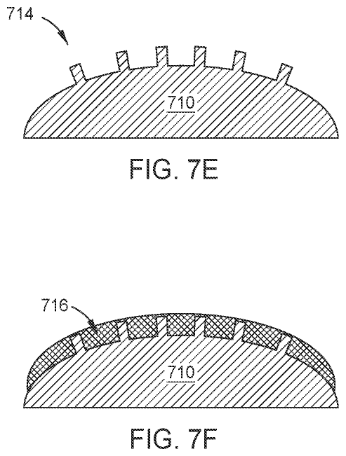

FIG. 6 depicts example operations 600 for forming each mold half 310, 312 (i.e., step 220) by soft lithography processes. Soft lithography can process a wide range of elastomeric materials (i.e., mechanically soft materials), such as polymers, gels, and organic monolayers. FIGS. 7A, 7B, 7C, 7D, 7E, and 7F illustrate aspects of a lens mold and a substrate corresponding to the different stages of various stages of the operations 600. As such, FIGS. 6, 7A, 7B, 7C, 7D, 7E, and 7F are described together.

At step 610, an elastomeric membrane (referred to as “replica mold” hereinafter) 702 is casted on the substrate 304 having the nanostructured pattern 302 formed thereon, as shown in FIG. 7A. The replica mold 702 may be formed of a thin flexible polymer, such as poly (dimethylsiloxane) (PDMS). In the casting process, PDMS in a liquid form may be mixed with a cross-linking agent and poured onto the substrate 304 and heated at an elevated temperature of between about 250° C. and about 350° C., hardening and cross-linking PDMS, to form the replica mold. The replica mold 702 may have a thickness of between about 200 μm and 500 μm. The replica mold 702 has protrusions 704 that replicates the nanostructured pattern 302 on the substrate 304. The protrusions 704 may have heights of between about 30 nm and about 200 nm, widths of between about 30 nm and about 300 nm, and spacings between adjacent trenches of between about 30 nm and about 300 nm. After casting, the replica mold 702 is removed from the substrate 304.

At step 620, a layer of photoresist 706 is spin coated on a curved surface 708 of a post 710, as shown in FIG. 7B. The curved surface 708 corresponds to the geometry of the outer surface 102A, 102P of the lens body 102. The photoresist 706 may be formed of polymeric material, such as polymethyl methacrylate (PMMA), and copolymer based on methyl methacrylate and methacrylic acid. The post 710 may be formed of material such as epoxy.

At step 630, a photomask, which is used to pattern the underlying photoresist 706 in the subsequent patterning step 640, is formed. To form a photomask, the replica mold 702 is placed on the photoresist 706, as shown in FIG. 7C, and patterned. The placement of the replica mold 702 on the photoresist 706 may be performed under vacuum in order to create suction between the replica mold 702 and the photoresist 706. Further, the patterning of the replica mold 702 may be performed by removing portions 712 of the replica mold 702 between adjacent protrusions 704 using electron-beam lithography (referred to as “e-beam lithography” or “EPL”). The patterned replica mold 702, having the protrusions 704, is used as a photomask in the patterning step 640, as discussed below.

At step 640, a patterned photoresist 706P, which is used to pattern the underlying curved surface 708 of the post 710 in the subsequent patterning step 650, is formed, as shown in FIG. 7D. To form the patterned photoresist 706P, the photoresist 706 is patterned using the photomask (the patterned replica mold 702, having the protrusions 704) formed at step 630, by photolithography. In the photolithography process, the photoresist 706 having the replica mold 702 disposed thereon is exposed to ultraviolet (UV) light, developed, and cured. Subsequently, the patterned replica mold 702 is removed. The patterned photoresist 706P is used in the patterning step 650, as discussed below.

At step 650, the curved surface 708 of the post 710 is patterned by non-isotropic etching, such as plasma dry etching, using the patterned photoresist 706P as a mask and a nanostructured pattern 714 is formed, as shown in FIG. 7E.

At step 660, a mold forming material precursor is sprayed onto the nanostructured pattern 714 of the curved surface 708 of the post 710 and cured, forming a mold 716 replicating the nanostructured pattern 302 on the substrate. This mold 716 can be used as a mold half 310, 312 having nanostructured pattern 316 (shown in FIGS. 3C and 3D).

System for Designing an IOL

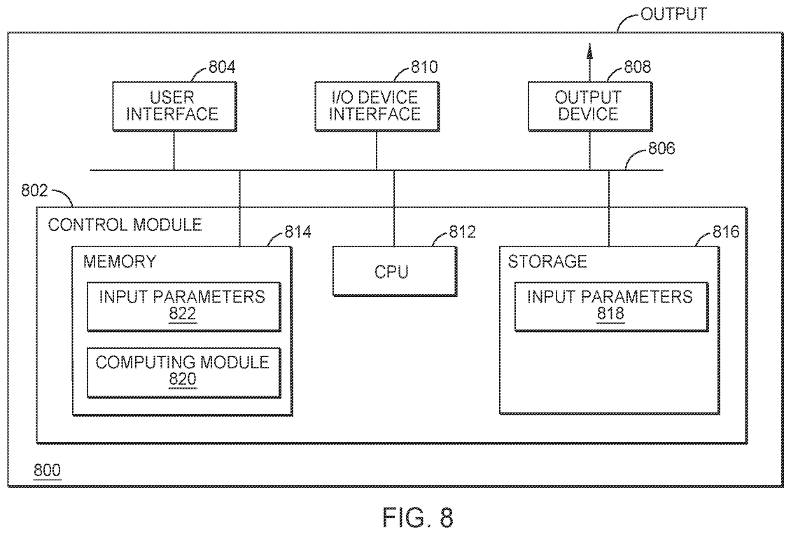

FIG. 8 depicts an exemplary system 800 for designing, configuring, and/or forming an IOL 100. As shown, the system 800 includes, without limitation, a control module 802, a user interface display 804, an interconnect 806, an output device 808, and at least one I/O device interface 810, which may allow for the connection of various I/O devices (e.g., keyboards, displays, mouse devices, pen input, etc.) to the system 800.

The control module 802 includes a central processing unit (CPU) 812, a memory 814, and a storage 816. The CPU 812 may retrieve and execute programming instructions stored in the memory 814. Similarly, the CPU 812 may retrieve and store application data residing in the memory 814. The interconnect 806 transmits programming instructions and application data, among CPU 812, the I/O device interface 810, the user interface display 804, the memory 814, the storage 816, output device 808, etc. The CPU 812 can represent a single CPU, multiple CPUs, a single CPU having multiple processing cores, and the like. Additionally, in certain embodiments, the memory 814 represents volatile memory, such as random access memory. Furthermore, in certain embodiments, the storage 816 may be non-volatile memory, such as a disk drive, solid state drive, or a collection of storage devices distributed across multiple storage systems.

As shown, the storage 816 includes input parameters 818. The input parameters 818 include a lens base power and a desired value of refractive index of a lens body. The memory 814 includes a computing module 820 for computing control parameters, such as configuration of the nanostructure assemblies 106A, 106P (e.g., shapes, sizes, and density). In addition, the memory 814 includes input parameters 822.

In certain embodiments, input parameters 822 correspond to input parameters 818 or at least a subset thereof. In such embodiments, during the computation of the control parameters, the input parameters 822 are retrieved from the storage 816 and executed in the memory 814. In such an example, the computing module 820 comprises executable instructions (e.g., including one or more of the formulas described herein) for computing the control parameters, based on the input parameters 822. In certain other embodiments, input parameters 822 correspond to parameters received from a user through user interface display 804. In such embodiments, the computing module 820 comprises executable instructions for computing the control parameters, based on information received from the user interface display 804.

In certain embodiments, the computed control parameters, are output via the output device 808 to a lens manufacturing system that is configured to receive the control parameters and form a lens accordingly. In certain other embodiments, the system 800 itself is representative of at least a part of a lens manufacturing systems. In such embodiments, the control module 802 then causes hardware components (not shown) of system 800 to form the lens according to the control parameters by the operations 200 described above.

Method for Forming an IOL

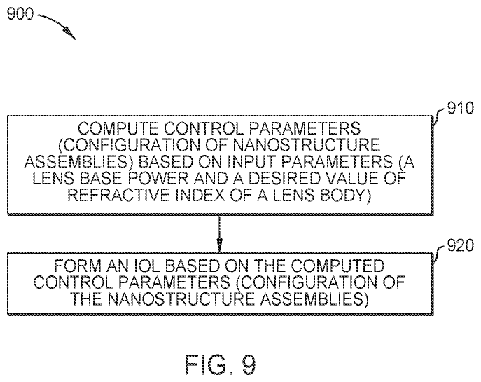

FIG. 9 depicts example operations 900 for forming an IOL 100. In some embodiments, the step 910 of operations 900 is performed by one system (e.g., the system 800) while step 920 is performed by a lens manufacturing system. In some other embodiments, both steps 910 and 920 are performed by a lens manufacturing system.

At step 910, control parameters, such as configuration of the nanostructure assemblies 106A, 106P (e.g., shapes, sizes, and density) are computed based on input parameters (e.g., a lens base power and a desired value of refractive index of a lens body). The computations performed at step 910 are based on one or more of the embodiments, including the formulas, described herein.

At step 920, an IOL (e.g., IOL 100) based on the computed control parameters, such as configuration of the nanostructure assemblies 106A, 106P (e.g., shapes, sizes, and density) is formed according to the operations 200 described above, using appropriate methods, systems, and devices typically used for manufacturing lenses, as known to one of ordinary skill in the art.

The embodiments described herein provide methods and systems for fabricating a plastic mold that allows for fabrication of a single piece monolithic IOL having nanostructured patterns embossed onto external surface(s) of the IOL, using injection molding. Therefore, the techniques described herein for creating a monolithic lens structure, with nanostructured patterns embossed on the external surface(s) thereof, simplify conventional fabrication processes. As discussed, conventional fabrication processes involve creating nanostructure patterns separately and attaching the nanostructured patterns to the external surfaces of an IOL, thereby resulting in higher complexity and manufacturing costs. Further, any risks associated with nanostructures detaching from an IOL in the human eye is eliminated, or at least reduced, with a monolithic lens structure having nanostructured patterns embossed on its external surface(s).

While the foregoing is directed to embodiments of the present disclosure, other and further embodiments of the disclosure may be devised without departing from the basic scope thereof, and the scope thereof is determined by the claims that follow.

1. An intraocular lens (IOL), comprising:
a monolithic lens body having an anterior nanostructure assembly on an anterior outer surface of the monolithic lens body and a posterior nanostructure assembly on a posterior outer surface of the monolithic lens body; and
one or more haptics coupled to the monolithic lens body.

2. The IOL of claim 1, wherein the monolithic lens body comprises biocompatible material.

3. The IOL of claim 1, wherein
each of the anterior nanostructure assembly and the posterior nanostructure assembly includes a plurality of protrusions having heights of between 30 nm and 200 nm, widths of between 30 nm and 300 nm, and spacings between adjacent protrusions of between 30 nm and 300 nm.

4. The IOL of claim 1, wherein
the monolithic lens body has a refractive index of between 1.49 and 1.56.

5. The IOL of claim 1, wherein
the anterior nanostructure assembly and the posterior nanostructure assembly are configured to reduce reflectivity of the monolithic lens body by between 10% and 90% as compared to a lens body with no nanostructure assemblies on the anterior outer surface or the posterior outer surface.

6. A lens mold for forming an intraocular lens (IOL) by injection molding, comprising:
a first mold half having a first inner surface, the first inner surface corresponding to an external geometry of an anterior outer surface of an IOL to be formed; and
a second mold half having a second inner surface, the second inner surface corresponding to an external geometry of a posterior outer surface of the IOL to be formed, wherein
the first inner surface has a first nanostructured pattern formed thereon, and
the second inner surface has a second nanostructured pattern formed thereon.

7. The lens mold of claim 6, wherein
the first mold half and the second mold half each comprises plastic material.

8. The lens mold of claim 6, wherein each of the first nanostructured pattern and the second nanostructured pattern includes a plurality of protrusions having heights of between 30 nm and 200 nm, widths of between 30 nm and 300 nm, and spacings between adjacent protrusions of between 30 nm and 300 nm.

9. A method for fabricating an intraocular lens (IOL), comprising:
fabricating a lens mold comprising a first mold half and a second mold half, wherein
the first mold half has a first nanostructured pattern formed on an inner surface of the first mold half, and the second mold half has a second nanostructured pattern formed on an inner surface of the second mold half; and
fabricating a single monolithic lens body using the lens mold by injection molding and thermal curing.

10. The method of claim 9, wherein
the first mold half and the second mold half each comprises plastic material.

11. The method of claim 9, wherein
the fabricating of the lens mold comprises:
spraying a mold forming material precursor over a substrate having a nanostructured pattern formed thereon; and
scanning the substrate by a focused ion beam.

12. The method of claim 11, wherein
the nanostructured pattern formed on the substrate extends over an area of 7 mm and 7 mm, and includes an array of trenches with heights of between 30 nm and 200 nm, widths of between 30 nm and 300 nm, and spacings between adjacent trenches of between 30 nm and 300 nm.

13. The method of claim 11, wherein
the mold forming material precursor comprises hydroxyl-groups-containing polypropylene (PP) precursor.

14. The method of claim 9, wherein
the fabricating the lens mold comprises:
casting a replica mold over a substrate having a nanostructured pattern formed thereon, the replica mold having a plurality of protrusions;
spin coating a layer of photoresist on a surface of a post;
placing the replica mold on the layer of photoresist;
patterning the layer of photoresist using the replica mold as a mask by photolithography;
pattern the surface of the post using the patterned layer of photoresist as a mask by plasma dry etching; and
spraying a mold forming material precursor on the patterned surface of the post and curing the mold forming material precursor.

15. The method of claim 14, wherein
the nanostructured pattern formed on the substrate extends over an area of 7 mm and 7 mm, and include an array of trenches with heights of between 30 nm and 200 nm, widths of between 30 nm and 300 nm, and spacings between adjacent trenches of between 30 nm and 300 nm.

16. The method of claim 14, wherein
the replica mold comprises poly(dimethylsiloxane) (PDMS).

17. The method of claim 14, wherein
the plurality of protrusions have heights of between 30 nm and 200 nm, widths of between 30 nm and 300 nm, and spacings between adjacent trenches of between 30 nm and 300 nm.

18. The method of claim 14, further comprising:
subsequent to the placing of the replica mold on the layer of photoresist, removing portions of the replica mold between adjacent protrusions of the plurality of protrusions by electron-beam lithography.

19. The method of claim 14, wherein
the mold forming material precursor comprises hydroxyl-groups-containing polypropylene (PP) precursor.

20. The method of claim 14, wherein
the post comprises epoxy.
